SEQUENCE LISTING (8 sequences)

SEQ ID NO: 1 - DNA, 21 bp, Homo sapiens
agctgaagaagatgtggaaaa

SEQ ID NO: 2 - DNA, 18 bp, Homo sapiens
agggactaggcgtgggat

SEQ ID NO: 3 - DNA, 25 bp, Homo sapiens
caaaaaggtaaaagcaaaggtaaaa

SEQ ID NO: 4 - DNA, 22 bp, Homo sapiens
aaggatggaaaagagaagaagg

SEQ ID NO: 5 - DNA, 20 bp, Homo sapiens
ccgctggtgatcaagcagga

SEQ ID NO: 6 - DNA, 24 bp, Homo sapiens
cacggtctgggcaagcctcgagat

SEQ ID NO: 7 - DNA, 25 bp, Homo sapiens
tcccagtccactatactgacgtctc

SEQ ID NO: 8 - DNA, 23 bp, Homo sapiens
tctctccatcctcatgttcttgg

(19) United States
(12) Patent Application Publication
(10) Pub. No.: US 2015/0283270 A1
(43) Pub. Date: Oct. 8, 2015

(54) MODELING ANTI-LEUKEMIC THERAPY BY PATIENT DERIVED AML XENOGRAFTS WITH DISTINCT PHENOTYPES/GENOTYPES

(71) Applicant: Crown Bioscience Inc., Santa Clara, CA (US)

(72) Inventors: Xiaoyu AN, Beijing (CN); Henry LI, Santa Clara, CA (US)

(73) Assignee: Crown Bioscience Inc., Santa Clara, CA (US)

(21) Appl. No.: 14/246,018

(22) Filed: Apr. 4, 2014

(51) Int. Cl. A61K 49/00; A01K 67/027

(52) U.S. Cl. CPC A61K 49/0008; A01K 67/0278; A01K 2267/0331; A01K 2207/12

(57) ABSTRACT

The present teachings relate to methods of screening for a therapeutic agent for human acute myelogenous leukemia (AML) and methods for treating AML. The method includes administering a test substance to an immunocompromised NOD/SCID mouse engrafted with a substance containing a leukemic cell derived from a human AML patient and a step of assessing improvement in leukemia in the mouse and/or to monitor a side effect of the test substance. The method can further include monitoring a side effect of the test substance in the mouse.

### FIELD

The present teachings relate to the use of patient derived xenographic mouse models for evaluating new chemotherapeutics and the treatment of patients with acute myelogenous leukemia.

### BACKGROUND

Acute myelogenous leukemia (AML) is a classic stem cell disease where self-renewing leukemic initiator cells (LICs) drive leukemogenesis of heterogeneous leukemic cell populations. The current models for AML are leukemia cell lines, e.g. HL60, THP-1, MV4-11, etc., which are maintained in vitro. With time in vitro cell lines become homogenous cell lines with undifferentiated phenotypes and are no longer distinct from either leukemic blasts or leukemic initiator cells (LICs) that are found in AML patients and are thus less reflective of the original disease. In addition, AML is not a single disease; patient-diversity cannot be reflected in in vitro AML cell lines.

Patient derived xenograft (PDX) has gained wide acceptance as the model of choice for studying oncogenesis and evaluating drugs in the solid tumor field since it has been shown to better represent the state of the original disease. However, establishing stable and serially transferrable AML engraftment has proven extremely challenging and hinders AML research and drug evaluation. The challenges include low level of engraftment (only detected at low levels in some organs such as BM), unstable, transient engraftment, low take-rate when serially transplanted or unable to be serially transplanted and asymptomatic or phenotype and genotype shift during growth and passages.

Chemotherapeutic agents used to treat AML can temporarily provide remission but there is no cure for AML and patients often relapse following cessation of chemotherapy. Uncovering the underlying pathways of leukemogenesis can lead to targeted therapeutics that could provide a cure of AML. Isocitrate dehydrogenase (IDH) mutations have been long expected to play a role in AML and other cancers (glioma, cholangiocarcinoma, etc.), and even been proposed to be a promising drug target for treating these diseases. However, due to lack of a suitable experimental model the oncogenic role of IDH has never been confirmed experimentally or its potential as a drug target it has never been validated.

Thus, understanding the pathways of leukemogenesis can be facilitated experimentally with animal models. AML animal models can advance the investigation of new chemotherapeutic agents as well as replicate human AML and also AML of individual patients. Furthermore, animal models having naturally occurring mutations (not artificially introduced), for example, IDH mutations, have potential biological (“efficacy”) readout to facilitate the confirmation of IDH as an oncogenic driver of leukemia and as a drug target for therapy.

Therefore, there remains a need to advance the investigation of new chemotherapeutic agents using AML animal models. The animal model can replicate human AML and also AML of individual patients. In particular, AML patients having mutations that reveal AML pathogenesis can rapidly advance the search for new treatment strategies against mutations as targets for chemotherapeutic agents. The use of animal models for screening new treatments and optimizing treatment strategies would be most advantageous to advancing survival of patients afflicted with AML as well as to advance the understanding of leukemic pathways in human.

### SUMMARY

In an embodiment disclosed is a method of screening for a therapeutic agent for human acute myelogenous leukemia. The method includes administering a test substance to an immunocompromised NOD/SCID mouse engrafted with a substance containing a leukemic cell derived from a human acute myelogenous leukemia patient and a step of assessing improvement in leukemia in the mouse. The method can further include monitoring a side effect of the test substance in the mouse. In one aspect the method includes examining the peripheral blood collected from the mouse to assess improvement in the leukemia and/or to monitor a side effect of the test substance.

In another aspect of the method the engrafted mouse reproduces the pathology of leukemia in the patient from whom the substance is derived. The mouse of the method can selectively expanded human leukemic cells, wherein the leukemic cells are leukemia initiation cells (LICs). The mouse of the method is a symptomatic leukemic mouse and exhibits at least one of an enlarged spleen or the presence of leukemic cells. The leukemic cells are present in the mouse of the method in at least one of bone marrow or a peripheral organ such as spleen or blood. The leukemic mouse of the method is an acute myelogenous leukemic mouse and engraftment of the mouse occurs when the mouse is about 4 weeks of age. The engrafted cells test positive for production of at least one of GM-CSF and IL-3.

In another aspect, the mouse of the method and its leukemic cells have the pathology of leukemia which replicates human AML subtype M0, M1, M2, M3, M4, M5, M6, or M7 based on the diagnosis of the human patient from which the engrafted leukemic cells were derived. The human patient can also have a heterozygous mutation. The mutation can be an IDH2 mutation such an R140Q mutation. The mutation can also be a heterozygous mutation of at least one of heterozygous FLT3-ITD, DNMT3A-R882H and NPM1

In another aspect disclosed is a method of selecting or optimizing a method of treating a patient from whom leukemic cells in the peripheral blood are derived from an immunocompromised NOD/SCID mouse engrafted with a substance containing a leukemic cell derived from a human acute myelogenous leukemia patient having a step of providing the mouse with a treatment of human acute myelogenous leukemia, and b) a step of assessing an improvement and/or a side effect caused by the treatment of leukemia in the mouse.

In yet another embodiment the mouse having human leukemic cells in peripheral blood is derived from a different non-adult immunocompromised NOD/SCID mouse about 4 weeks old having one or more repeats of a step of engrafting a substance containing a leukemic cell derived from a first mouse obtained by engrafting the first mouse with a substance containing a leukemic cell derived from a human acute myelogenous leukemia patient and raising the different mouse, wherein the different mouse has human leukemic cells in at least one of the mouse peripheral blood, bone marrow or spleen.

In still yet another embodiment disclosed is a method of producing a mouse having human leukemic cells, comprising engrafting a substance containing a leukemic cell derived from a human acute myelogenous leukemia patient into a non-adult immunocompromised NOD/SCID mouse and raising the mouse.

In yet another embodiment disclosed is a method of producing a mouse having human leukemic cells, comprising one or more repeats of a step of engrafting a substance containing a leukemic cell derived from the mouse obtained by engrafting a substance containing a leukemic cell derived from a human acute myelogenous leukemia patient into a non-adult immunocompromised NOD/SCID mouse into a different non-adult immunocompromised NOD/SCID mouse and raising the mouse.

In any of the embodiments described above, the methods can include examining the peripheral blood collected from the mouse about four weeks after engraftment, or at any time after administering to the engrafted mouse a treatment of AML or to determine if a treatment has a toxicological affect or if a treatment has not therapeutic impact on the leukemic cellular load in the mouse's peripheral blood, bone marrow or spleen or other organs such as the head, kidney, or lung. The methods can also include examining the engrafted cells for production of at least one of GM-CSF and IL-3 as well as chemokines and cytokines.

In any of the embodiments described above, the methods the engrafted mouse and the leukemic cells derived from the engrafted mouse reproduces the pathology of leukemia in the patient from whom the substance containing a leukemic cell derived from a human AML patient is derived. The engrafted mouse also reproduces the phenotype and genotype of the leukemia cells in the patient from whom the substance containing a leukemic cell derived from a human AML patient is derived. Additionally, the engrafted cells can be selectively expanded human leukemic cells, which are obtained by one or more repeats of a step of engrafting a leukemic cell derived from a human AML patient into a non-adult immunocompromised NOD/SCID mouse and raising the mouse. Such a mouse is known as a leukemic mouse and exhibits at least one of an enlarged spleen or the presence of leukemic cells. The leukemic cells are present in at least one of bone marrow or a peripheral organ such as spleen or blood. Additionally, the leukemic mouse is an acute myelogenous leukemic mouse and the leukemic cells are leukemia initiation cells.

In any of the embodiments described above, the leukemic cell can be a mononuclear cell, wherein the mononuclear cell is extracted from a tissue in the patient's bone marrow, spleen or blood and the extracted cell is obtained by Ficoll® column separation. The engraftment results in a high percentage of leukemic cells seen in bone marrow as well as in peripheral organs such as at least one of the blood and spleen.

### BRIEF DESCRIPTION OF THE DRAWINGS

FIG. 1. A: Illustration, as determined by flow cytometry, of the leukemic load defined as percentage of human CD45⁺cells in total peripheral blood cells of AM7577-mice of different passages (P0-P3), which was quantified weekly post-engraftment; B: Illustration of the survival curves of AM7577-mice of different passages; C: Depicts a photograph of a microscopic image of a leukemia cell smear by Wright-Giemsa Stain (100×); D: Depicts a photograph of an enlarged spleen of a AM7577-mouse; E: Illustrates immunological phenotypes as determined by flow cytometry using leukemia cells harvested from AM7577-P0 mice. Leukemic cells exhibit CD38⁺, CD123⁺, CD45⁺, CD13⁺, CD33⁺; CD34⁻, HLA⁻, DR⁻, CD19⁻; F: Graphical representation of plasma concentration of 2HG at different time post-engraftments and percentage of CD45+ cells; G: Depicts microscopic photographs of hCD45− IHC analysis of internal organ tissues of AM7577-mice: infiltration of leukemic cells.

FIG. 2. FIGS. 2A-2D results determined by using plasma of AM7577-mice in a cytokine-specific ELISA A: Bar graft illustrating the secretion by AML cells of hIL-3; B: Bar graft illustrating the secretion by AML cells of hGM-CSF; C: Bar graft illustrating hIL-3 autocrine signaling in AM7577-mice; D: Bar graft illustrating hGM-CSF autocrine signaling in AM7577-mice; E: Depicts a photograph of CFCs in leukemia cells isolated from spleen of AM7577-mice; F: Bar graft representation of the impact on the presence of cytokines on CFC formation in leukemia cells; G: Table representing plasma concentration of 2HG at different time points post-engraftment.

FIG. 3. Illustrations of Cytarabine anti-tumor activity in AM7577-mice. A: Leukemic burden as % of hCD45+in the peripheral blood ofAM7577-mice treated with Cytarabine (Ara-C) 15 days after engraftment with 3 million leukemia cells harvested from P3, different doses of Cytarabine were administered in 6 mice (5 days on, 2 days off) as duration indicated in the figure. There are two periods of dosing. The first one lasted for 8 weeks and the second one for 2 weeks; B: Survival curves under Cytarabine treatment; C: Leukemia burden in different organs under the treatment.

FIG. 4. Illustrates treatment of AMM7577-mice by various chemotherapeutics. Inhibition of leukemia in blood by AC220 (A); Inhibition by Sorafenib (B); survival by AC220 and Sorafenib (C) treatment.

FIG. 5. A: Illustration of the presence of the IDH2 R140Q mutation in the AML PDX mice; B: IDH2 mutation analysis. Lane 1=Wildtype; Lane 2=AMM7577-P0; Lane 3=AMM7577-P1; Land 4=AMM7577-P2.

FIG. 6. Illustrates leukemia development kinetics with different numbers of leukemic cells per inoculation.

### DESCRIPTION OF VARIOUS EMBODIMENTS

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “a leukemic” includes one or more leukemic cells, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure. All publications mentioned herein are incorporated herein by reference in their entirety.

Reference will now be made to various embodiments, examples of which are illustrated in the accompanying drawings.

The present teaching can be applicable to any hematological disease characterized by abnormal cellular proliferation or solid tumor formation. Acute myelogenous leukemia (AML) is the most common adult acute leukemia with increasing incidence as the population ages. AML is characterized by accumulation of immature, abnormal hematopoietic cells in the bone marrow and also in the peripheral blood in many cases. AML has several subtypes with varying prognosis, including M0 to M7 per FAB (France-American-British) classification. The five-year survival rate varies from 15% to 70%, and relapse rates vary between 33% and 78% depending on the subtype. Most common treatments are chemotherapies that can cause remission in many cases but without cure.

Lack of a predictive experimental model of AML has been an obstacle for understanding leukemogenesis and the search for effective treatments. The challenges to creating a sustained xenograft leukemic state in mice derived from AML patients could be two fold: First, the residual immunity in the immunocompromised mice which prevents engraftment; and Second, lack of a suitable environment to support AML leukemogenesis in mice. AML cell growth requires the presence of human cytokines, including GM-CSF and IL-3.It is known that mouse cytokines do not cross interact with human receptors on the leukemic cells. To overcome these obstacles, technological innovations have been developed to facilitate AML engraftment. Mouse strains with further immune-compromise in its immunity, including enhanced reduction of natural killer activity (NK) in NOG, NOD/SCID/IL-2Rc null mice (NSG) (Malaise M, et al., Leukemia. 2011 (25):1635-9 and Shultz L D, et al., J. Immunol. 2005 (174):6477-89) or pre-conditioning such as irradiation, can help to overcome the first obstacle. In fact, accelerated leukemogenesis was reported in NSG (Agliano A, et al., Int. J. Cancer, 2008 (123):2222-7). On the other hand, “humanized mice” expressing a transgenic human cytokine gene, e.g. IL-3, GM-CSF, is another method to overcome the second obstacle (Feuring-Buske M, et al., Leukemia. 2003 (17):760-3. Several factors have also been reported to be associated with higher engraftment, including poor patient prognosis (Lumkul R, et al., Leukemia. 2002 (16):1818-26; Pearce D J, et al., Blood, 2006 (107):1166-73 and FLT3 mutation (Rombouts W J, et al., Leukemia, 2000 (14):889-97; Rombouts W. J., Blokland I., et al., Leukemia. 2000 (14):675-83), although with controversy (Pearce D J, et al., supra).

In one embodiment disclosed is a newly established model of AML-M5 in NOD/SCID mice by engrafting AML patient leukemic cells. The model reflects aspects of AML-M5 disease in symptoms and pathology and its leukemic cells maintained both phenotype and genotype of the original patient leukemic cells. Several unique characteristics of this model distinguished it from many other reported models including the robust and reproducible growth of leukemia to very high levels, stable transferability (100%) in serial transplantation, offering an unlimited and consistent source of primary AML cells. The unique success to establish AM7577 could result from overcoming the second barrier described above—an autocrine mechanism of cytokine/receptor signaling. We believe it can be a useful experimental model for exploring leukemogenesis and also for evaluating new investigational therapies for AML.

Several genetic mutations are frequently associated with AML, among which, some could be oncogenic drivers and thus serve as targets for new therapies. Somatic mutations in isocitrate dehydrogenase 1 (IDH1, cytosolic) and IDH2 (mitochondrial homolog) have also often been associated with AML (7% and 15% respectively), as well as other malignancies, e.g. gliomas, etc. (Dang L, et al., Trends Mol Med. 2010 (16):387-97; Zhou K G, et al., Leuk Lymphoma. 2012; 53(12): 2423-9). IDH mutations lead to 2-hydroxyglutarate (2HG) production in leukemic cells, which can be detected both in leukemic cells and plasma of AML patients. This association has led to speculation that IDH mutations are important in AML leukemogenesis (an induction or production of leukemia), and could also potentially served as drug targets for AML. However, these assumptions have never been verified for lack of a suitable experimental model, which also prevents developing IDH targeting therapy. While there is no naturally isolated leukemic cell line with an IDH mutation, the artificially introduced IDH mutations in engineered cell lines have failed to demonstrate oncogenic phenotypes that can be used as reports for measuring anti-tumor effects or testing potential chemotherapeutic treatments for AML.

IDH mutations have long been expected to play role in AML and other cancers (glioma, cholangiocarcinoma, etc.), and have even been proposed to be a promising drug target for treating these diseases. However, due to the lack of a suitable experimental model, its oncogenic role has never been confirmed experimentally. As a potential drug target it has never been validated. This disclosure, to our knowledge, is the first reported AML model, or cancer model for that matter, that has an intrinsic IHD mutation (not artificially introduced) and thus has the potential biological (“efficacy”) readout to facilitate the confirmation of IDH as an oncogenic driver and a drug target for therapy. Testing an IDH mutation inhibitor in an AM7577 mouse could potentially accomplish IDH as an oncogenic driver and as a potential drug target.

Thus, experimental models reflecting an original human disease with an IDH mutation as seen in patients, including AML patients, would have value in advancing understanding of leukemogenesis and developing new cancer treatments. Therefore, AM7577 could serve as a useful model to investigate the roles of IDH2mutations in AML pathogenesis and search for new treatment strategies targeting this mutation.

2-hydroxyglutarate (2HG) is a metabolite characterizing an IDH mutation. 2HG was found in leukemic cells and plasma of AM7577 mice as readily measured by HPLC/MS with 2HG levels correlating to its leukemic loads in blood. A confirmation that the AM7577mice were phenotypically AML was the production by the leukemic cells of GM-CSF and IL-3, cytokines required for myelogenous development and AML cell growth, as determined by either ELISA or functional assays. This presence of GM-CSF and IL-3 in the leukemic cells of the AML mice suggests that an autocrine mechanism may play a key role in the robust leukemic cell growth in mice. In addition, colony formation units (CFUs) in leukemic cell populations were measured in semi-liquid cultures in the absence of these two cytokines, which, together with serial transplantability of the leukemic cells, supports the presence of leukemia initiation cells (LICs). The treatment of AM7577 mice with AraC, a standard chemotherapy for M5-subtype AML, resulted in complete remission (below detection levels in blood) and extended life. Leukemia, however, rapidly relapsed shortly after AraC withdrawal, suggesting that AraC, although effective against leukemoblasts, has little effect on LICs.

Disclosed are the design and development of a mouse model for AML. The transplantation of bone marrow (BM) cells of an AML-M5 patient (AM7577) having a heterogenous IHD2 mutation into immunocompromised NOD/SCID mice resulted in a robust and stable engraftment of the BM cells, leading to full-blown AML and mortality of the mouse from AML. The engraftment can serially be passed from the primary mouse to other immunocompromised NOD/SCID mice with 100% take-rate of the xenografts. The engraftment maintains all the genotype and phenotypes of the patients, including the IDH2 R140Q mutation (Table 1).

TABLE 1
Summary of AM7577 patient information
and mouse model information

| | Patient | AML Mouse Model |
|---|---|---|
| | Male, 69-yrs old, M5-subtype | |
| Chromo- | Normal karyotype | |
| some | | |
| Blood-Rt. | WBC 38.9X109/L, HB 74.2 | |
| | g/L, plt 163X109/L, abnormal: | |
| | 79.20%, Naive/nuclear: 11.47%. | |
| | Classification of blood: 63% | |
| Mutations | FLT3(+); DNMT3A(+); | FLT3(+); DNMT3A(+); |
| | IDH2(R140Q); NPM/A, | IDH2(R140Q); CEBP- |
| | (SNP: CEBP-2′ + ins c) | 2′ + ins c; NPM/A |
| Immuno- | Expresses: CD13, CD33, HLA- | Expresses: CD38, |
| logical | DR, CD117, CD38, CD71. | CD123, CD45, CD13, |
| Phenotype | Partially expresses: MPO, | CD33 |
| | CD15, CD19, CD7. | Does not express: |
| | Does not express: CD34, | CD34, HLA-DR, CD19 |
| | CD10, CD20, CD79a, CD3, | |
| | CD5, CD11b, CD14, CD56, | |
| | GlyA | |
| Treatment | Chemotherapy (ECAG), | Complete responses to |
| & response | complete response | AraC, sorafenib & |
| | | AC220. No relapse for |
| | | AC220 after drug |
| | | withdrawal |

### General Definitions

The term “about four weeks old” as used herein refers to a non-mature rodent being at least 28 days of age. That is, the rodent can be 29, 30, 31, 32 or 33 days of age or 28.5 days of age.

The phrase “amount of the human disease impacted by the agent” as used herein refers to at least one of the growth of the tumor, weight of the tumor, size of the tumor, weight of the rodent who received the agent as a treatment or screen for a treatment for a human disease and the like as a way to access the efficacy of a human disease treatment agent. As used herein “agent” refers to a biologic or a chemical treatment for a human disease or infection.

The phrase “human immune cell lineages” as used herein refers to cells exhibiting cell surface immune cell markers. The human immune cells can express immune markers, including but not limited to, CD34, CD16, HLA-DR (MHC II), CD14, (monocytes), CD11c (Dendritic cells), CD11b (Neutrophilic cells), CD19 (B-cells, early stage), CD20 (B-cells, late stage), CD3 (T-cells), CD56 (NK cells) and CD33 (Myloid cells).

The phrase “monitoring a side effect of the test substance” as used herein refers to evaluating toxicity, teratogenicity, and other effects a test substance, such as a chemotherapeutic agent, a biologic or a compound or a treatment can have upon a model organism, an in vitro cell culture or other in vitro or in vivo testing systems to determine both efficacy and efficiency but also deleterious or undesirable effects of a test substance. Evaluations can include but are not limited to gross examination of the model system as well as extracting and analyzing blood or blood components, X-ray, dissection and analyses of tissues, organs and cells and cell preparations from the model organism, cell culture or testing system.

The phrase “positively impacts the amount of the human disease in the human disease-infected mouse” as used herein refers to a clinically measurable or quantifiable presence of disease (or infection). The disease would be reduced as measured by tumor size, reduced circulating immune lineage cells, decreased disease (tumor presence) pathology as seen in tissue biopsy and staining and the like as would be known to one of skill in the art.

The phrase “reproduces in the mouse the pathology of disease in the patient from whom the substance is derived” as used herein refers to a PDX mouse having a human disease of the same phenotype and genotype as the human patient from which were extracted the diseased cells that were then transplanted into the mouse.

The term “a substance containing a leukemic cell” as used herein refers to a liquid containing a leukemic cell extracted from at least one of bone marrow or peripheral blood. During extraction of the leukemic cell, both plasma and other cells types can be extracted and may remain in the substance containing the leukemic cell. Cell types can include but are not limited to mononuclear cells, which include leukemic initiator cells, also known as leukemic stem cells, osteoclasts, white blood cells and red blood cells. The leukemic cell extracted can be further purified by, for example, passing the leukemic cell extract solution through a Ficoll® column to enrich for the leukemic cell population. It is the enriched leukemic cell population that is contained within the fluidic substance. The substance can be used for injection into a rodent to establish a xenograft of leukemic cells in the recipient rodent.

The term “selectively expanded human leukemic cell” as used herein refers to the propagation of human leukemic cells by engraftment of human leukemic cells into a rodent host. The leukemic cells can grow predominantly in the bone marrow, peripheral blood or spleen but can also be detected in the liver and lung and possibly kidney of the engrafted rodent. The current disclosure provides significant take-rate and full engraftment in a rodent host of human AML leukemic cells. Testing for CD45+ cells confirmed the high take-rate and further supporting that the AML cell was expanded selectively in the human patient derived xenografted rodent.

The term “human leukemic cells” as used herein refers to cells exhibiting phenotypic and genotypic characteristics of human AML leukemic cells. Depending on the FAB (France-American-British) classification, the human leukemic cell can express myeloid markers, including but not limited to, CD45, CD33, CD123, and CD13, along with negative staining for non-myeloid markers, e.g. CD19.

The term “leukemic mouse” as used herein refers to a mouse having symptoms of leukemia. It can also refer to a patient derived xenograft (PDX) mouse in which leukemic cells extracted from a human AML patient are transplanted into a mouse and the mouse develops AML and also reproduces the phenotype and genotype of the patient AML leukemic cells that were transplanted.

The term “substance” as used herein refers to a solution containing either human tissues or human immune progenitor cells such as mononuclear cells (MNC's), human stem cells, or human stem cells capable of developing into human immune cell lineages such as CD34+ cells or tumor cells derived from a diseased or infected human or mouse. The tumor tissues or tumor cells can be of hemopoiesis origin, or a malignant neoplastic tumor. The tumor can be but is not limited to an epithelial tumor or a tumor of lung, breast, gastro-intestinal including colon origins. The cells can be suspended in PBS, saline or another vehicle as is known to one of skill in the art for injecting intravenously or intraperitoneally cells for engraftment.

The term “reproduces in the mouse the pathology of leukemia in the patient from whom the substance is derived” as used herein refers to a PDX mouse having leukemia of the same phenotype and genotype as the human patient from which were extracted the leukemic cells that were then transplanted into the mouse.

### EXAMPLES

Mice and Engraftment Procedure

Animal experiments were conducted at Crown Bioscience's laboratory, China. The transplantation recipient NOD/SCID mice were purchased from Beijing HFK Bioscience at between 3-4 weeks of age. The Institutional Animal Care and Use Committee (IACUC) of Crown Bioscience approved animal study protocols. All procedures were under sterile conditions at Crown Bioscience's SPF facility and conducted in strict accordance with the Guide for the Care and Use of Laboratory Animals of the National Institutes of Health. Ficoll® column isolated MNCs were injected into the tail-vein of 4-week old, sub lethally irradiated NOD/SCID mice (1.5Grad), followed by weekly monitoring for peripheral appearance of leukemic cells (CD45⁺) via retro-orbital bleeding and flow analysis. Briefly, ˜50 μL blood was collected into anticoagulative tubes (BD 365974, Becton Dickinson and Co., Franklin Lakes, N.J.). After lysis of red blood cells the cells were stained with 20 μL mouse anti-human CD45 antibody (Biolegend, CA) and incubated on ice for 30 minutes in the dark followed by washing with cold PBS. The cells were resuspended in150 μL PBS and subjected to FACS analysis.

Patient Samples

All procedures were approved by the Institutional Review Boards (IRB) of the Wuhan Tongji Hospital and with the informed consent from the patients. Bone Marrow (BM) was harvested through BM aspiration from an AML patient at Wuhan Tongji Hospital (China) for patient diagnosis, including immunological phenotyping and genotyping. Mononuclear cells (MNCs) were isolated immediately from the BM sample via Ficoll® separation (HISTOPAQUE-1077, Sigma-Aldrich). A sample fraction of MNCs was used for engraftment.

Cell Proliferation Assay and Colony Formation Assay

Colony formation cells (CFC) were grown in MethoCult® Media (STEMCELL Tech, Hanzhou, Zhejiang, 310012, CN) in the presence of a human cytokine mix of SCF and IL-6 (NGI) per the manufacturer's instruction. Briefly, AM7577 spleen cells from either cryopreserved frozen vials (or freshly isolated from a leukemia mouse) were washed with MethoCult® Cell Wash Medium (10 minutes, 2 washes), and then adjusted to a final concentration of 2×10⁶ cells/mL in the same media. 0.1 mL of cells were mixed with 0.9 mL Methylcellulose Media containing 2 uL SCF-1 and IL-6 and then an aliquot of 0.5 mL was dispensed into each well of a 12-well plate. The cells were incubated at 37° C. for 7-14 days, in the presence of 5% CO₂ and ≧95% humidity.

Evaluation of Anti-Leukemia Activity of a Therapeutic

Fifteen days post-engraftment, mice were administered different doses of cytarabine (AraC). Mouse blood was collected by retro-orbital bleed and human CD45⁺ cells were quantified weekly by flow cytometry. Body weight and clinical signs were also monitored twice weekly. When mice became morbid due to full-blown leukemia, the animals were sacrificed, followed by gross and microscopic examinations. Major relevant organs (bone marrow, spleen, blood, liver, lymph nodes, etc.) were examined and harvested for further analysis, including IHC (method described previously, Yang, M. et al., Int J Cancer. 2012; Sep. 5. doi: 10.1002/ijc.27813) and immunological phenotyping of the leukemia cells by FACS.

Mutation Analysis

Hotspot mutation analysis was performed according to the method of Yang, M. et al., supra.

### PCR Primers Used:

| Mutation | | SEQ ID |
|---|---|---|
| Sequence | Primer | NO: |
| IDH2-F | AGCTGAAGAAGATGTGGAAAA | 1 |
| IDH2-R | AGGGACTAGGCGTGGGAT | 2 |
| FLT3-EXON14-F | CAAAAAGGTAAAAGCAAAGGTAAAA | 3 |
| FLT3-EXON14-R | AAGGATGGAAAAGAGAAGAAGG | 4 |
| CEBPA-2-F | CCGCTGGTGATCAAGCAGGA | 5 |
| CEBPA-2-R | CACGGTCTGGGCAAGCCTCGAGAT | 6 |
| DNMT3A-F | TCCCAGTCCACTATACTGACGTCTC | 7 |
| DNMT3A-R | TCTCTCCATCCTCATGTTCTTGG | 8 |

2HG Analysis

2HG was assayed using 10 μL plasma. 90 μL acetonitrile containing internal standards was added to 10 μL of plasma followed by stirring and centrifugation. The 90 μL supernatant was mixed with 90 μL water and then injected into an LC-MS/MS for analysis (Acquity Df UPLC-API4000 LC-MS/MS System, AB Sciex). To assay 2HG in leukemic cells 450 μL 50% MeOH was added to a50 μL cell sample, followed by thorough shaking to yield cell lysates. 450 μL acetonitrile containing internal standard was then added to the 50 μL cell lysates, followed by 1:1 dilution prior to injection into the LC-MS/MS for analysis.

Statistical Analysis

The data for leukemic load were evaluated using Student's t-test for two comparisons, and one-way ANOVA test for multiple comparisons. All data were analyzed using GraphPad Prism 5.0 software (GraphPad Software, Inc. La Jolla, Calif.). Values of P<0.05 were considered to be statistically significant.

Standard Immunohistochemistry, Western Blot and ELISA Analyses

Standard immunohistochemistry (IHC) and Western blot analysis were used to analyze tumor tissues as described before (Yang M. et al. supra). ELISA assays of GM-CSF and IL-3 were performed according to the products' protocols (Nanjing Tianwei Biotech, Nanjing, China). Briefly, 10 μL of plasma was diluted into 40 μL of dilution buffer and added into the ELISA plate provided by the vendor. After incubating for 30 minutes at 37° C. degrees, the plate was washed twice with buffer. 50 μL of HRP-conjugated reagent was added into each well and absorbance was read at 450 nm.

### EXAMPLES

### Example 1

### Engraftment of AML Patient BM Cells in NOD/SCID Mice

Bone marrow (BM) of Patient-AM7577 was harvested at Tongji Hospital for diagnosis. The BM sample was subjected to pathology analysis, immunological analysis, cytogenetic analysis and genetic mutation analysis with the results shown in Table 1. The cytogenetic and pathology analyses revealed that this patient, had FAB-M5-subtype AML with normal karyotype. The disease progressed rapidly. The patient was treated with ECAG, several cycles of regimens: VP-16, etoposide; CAG, low-dose Cytarabine and Aclarubicin in combination with granulocyte colony-stimulating factor. Following initial response to standard of care of chemotherapy, disease rapidly recurred, and the patient subsequently died of the disease.

Immunological analysis using flow cytometry revealed that AM7577 patient BM cells were positive staining for typical myeloid surface markers of CD45, CD33, CD123, and CD13, along with negative staining for non-myeloid markers, e.g. CD19, and thus confirmed the AML diagnosis (Table 1). The leukemic cells were also subjected to hot-spot mutation analysis, and results confirmed that the leukemic cells contain heterozygous IDH2 R140Q mutation, along with mutations of FLT3-ITD, DNMT3A-R882H and NPM1 (Table 1). These mutations are commonly associated with typical AML.

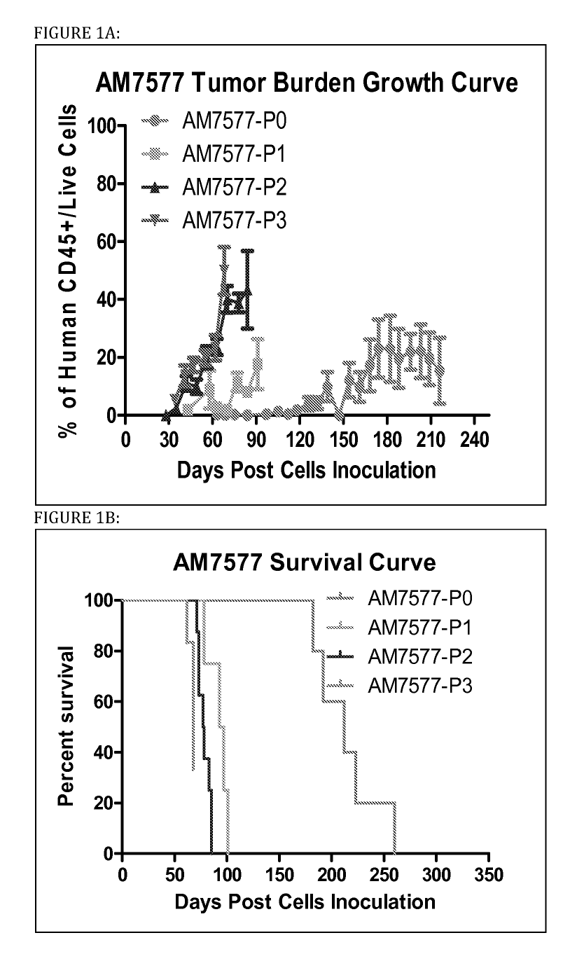

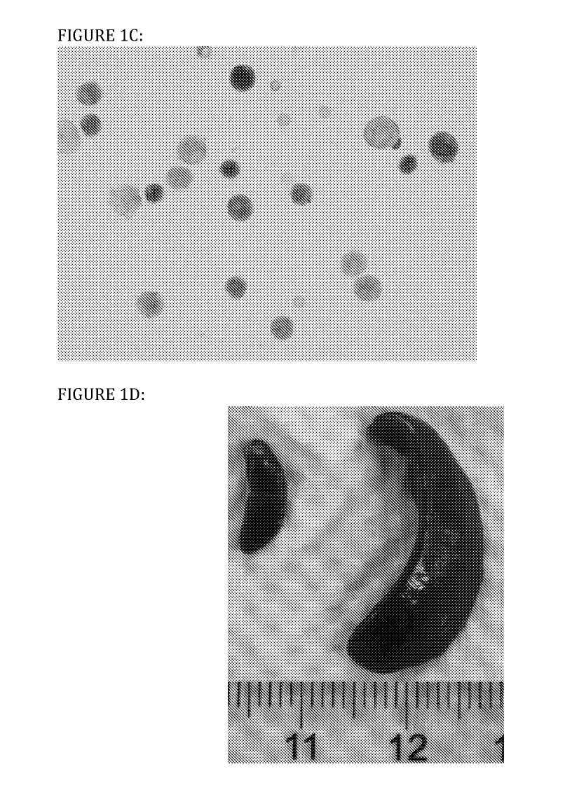

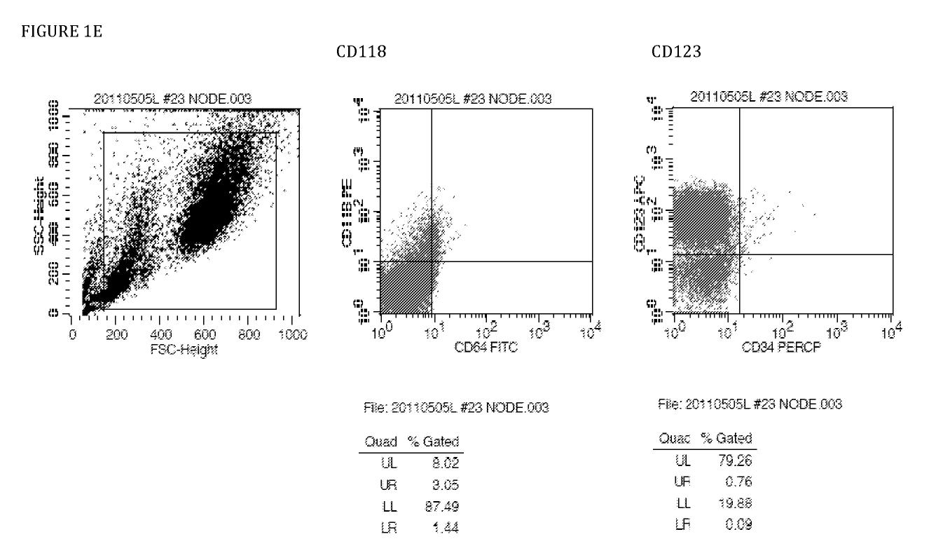

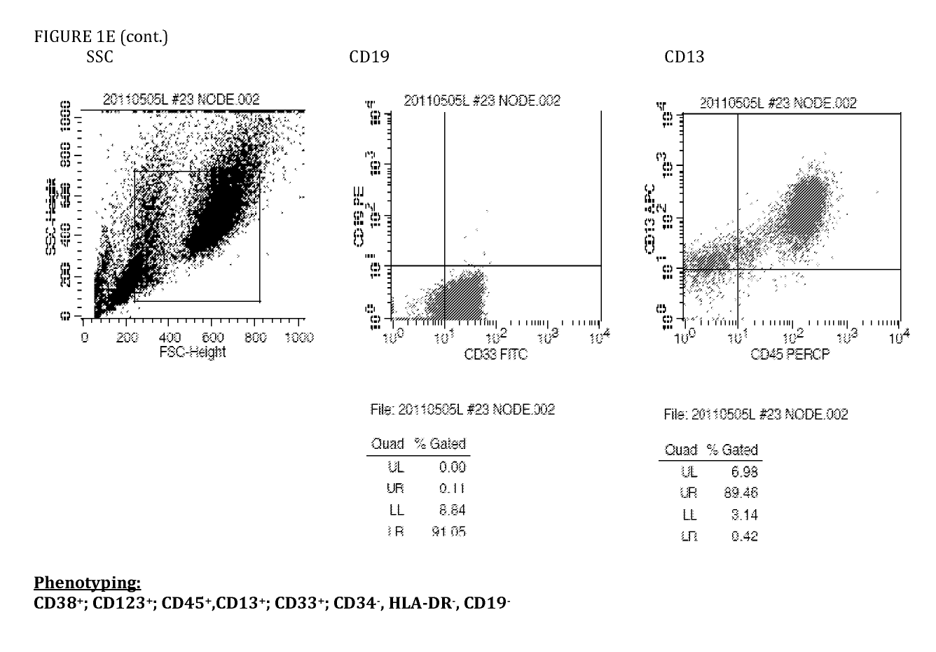

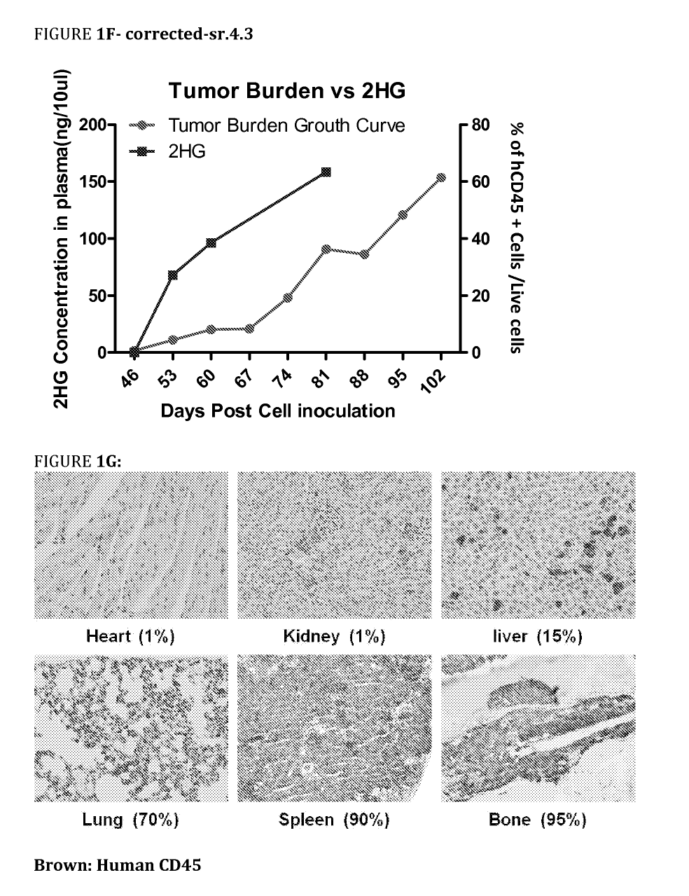

Mononuclear cells (MNCs) derived from AM7577-BM were engrafted into five 4-week old sub lethally irradiated female NOD/SCID mice in order to create patient derived xenograft (PDX) models by intravenous injection. The transplanted animals were monitored by weekly bleed and FACS analysis for hCD45⁺ cells in peripheral blood. All five AM7577-MNC transplanted mice (P0) showed leukemic cell growth in peripheral blood (FIG. 1A) with relative long latency (5 months). AM7577 mice ultimately developed full-blown AML (FIG. 1A) accompanied with typical symptoms of body weight loss, hunched, inactivity, labored breathing, ruffled coat and eventual morbidity/mortality (100%, FIG. 1B).

### Example 2

### Characterizations of AM7577-Mice

High engraftment levels in different animal organs. Not only were the MNCs of patient AM7577 found to have a high take-rate of 100% for engraftment, but they also displayed particularly high levels of engraftment. High leukemic loads as measured reflected this by flow cytometry monitoring of hCD45⁺ cells in peripheral blood (up to >70%) (FIG. 1A) and in other hemopoietic organs including BM (up to 99%) (Table 3), spleen (up to 95% leukemic cells in the significantly enlarged spleen) (FIG. 1D, Table 3), and lymph node. Leukemic cells also infiltrate other internal organs including spleens, liver and sometimes brain, as commonly seen in M5 patients. This level of AML engraftment was characteristically high, in contrast to many other reported engraftments. The representative FACS analyses of leukemic cells from these organs are shown in FIG. 1E. In addition, immunohistochemistry (IHC) was also used to confirm the infiltration of leukemic cells into different organs, including liver, lung, bone and spleen, as shown in FIG. 1F.

TABLE 3
Leukemic load in bone marrow and spleen AM7577 model

| | | % hCD45⁺ | | Total hCD45⁺ (10e6) | |
|---|---|---|---|---|---|
| Passage | animal # | Spleen | BM | spleen | BM |
| P0 | 20110505L #23 | 76.93 | 86.73 | 28 | N/A |
| | 20120214G #13 | 75.23 | N/A | 21 | N/A |
| | 20120214G #14 | 78.51 | N/A | 21 | N/A |
| | 20120214G #15 | 66.06 | N/A | 18 | N/A |
| P1 | 20111122E #1 | 92.84 | 99.21 | 139 | 7 |
| | 20111122E #2 | 69.91 | 98.97 | 32 | 9 |
| | 20111122E #3 | 37.38 | 99.21 | 35 | 9 |
| | 20111122E #4 | 48.84 | 88.21 | 57 | 9 |
| | 20111122E #6 | 73.73 | 98.28 | 78 | 22 |
| P2 | 20120118D #3 | 73.98 | 97.84 | 115 | 39 |
| | 20120118D #4 | 72.83 | 97.78 | 80 | 15 |
| | 20120118D #5 | 85.78 | 98.4 | 232 | 25 |
| | 20120118D #6 | 77.76 | 98.93 | 170 | 20 |
| | 20120118D #8 | 48.33 | 97.57 | 53 | 26 |
| | 20120118D #9 | 73.09 | 97.63 | 121 | 21 |
| P3 | 20120410K 11# | 72.55 | N/A | 163 | N/A |
| | 20120410K 12# | 68.27 | 99.57 | 76 | 7 |
| | 20120410K 15# | 46.03 | 96.74 | 126 | 5 |
| | 20120410K 19# | 95.42 | 85.78 | 112 | N/A |

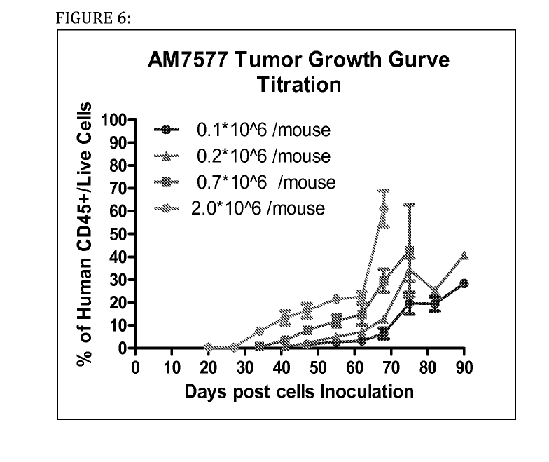

Serial transplantation of AM7577. The leukemic cells can be serially passed via re-engraftment of recipient mice using BM or spleen MNCs (even with <1×10⁵ cells) from donor leukemic AM7577-mice with 100% take-rate. The re-engraftments (P1, 2, 3, . . . ) all have similar growth kinetics (FIG. 1A) and caused 100% leukemia induced mortality (FIG. 1B). Therefore, serial transplantation has been shown to consistently create a robust and stable AML model. This model also provides a renewable and potentially unlimited source of primary leukemia cells. We have tested different numbers of cells for inoculation, from 1×10⁵ to 3×10⁶ cells, and found the take-rate is 100% regardless of the number of cells inoculated. The only difference observed was the onset of disease: The more cells inoculated, the faster the leukemia developed (FIG. 6). The faster onset is likely due to higher numbers of LICs from a higher total number of leukemic cells present in the inoculum. Slight acceleration of leukemia onset was also observed with an increase in passage number, a phenomenon also reported by others (Agliano A, et al., Leukemia, 2011 (25):1635-9). This could also potentially be explained by further enrichment of LICs in the leukemic cell population during the process of passage.

Phenotypic and genotypic characterization of AM7577 leukemic cells. We have comprehensively characterized the leukemic cells in the AM7577 models. Wright-Giemsa staining of BM leukemic cells display typical AML leukemic cell morphology (FIG. 1C). Flow cytometry analysis showed that leukemic cells from the models are identical to those of the original patient leukemic cells in the immunologic markers detected, including CD45⁺, CD33⁺, CD13⁺, CD123⁺, CD38⁺, and CD19⁻, CD34⁻, HLA-DR⁻, CD19⁻ (FIG. 1E) (Table 1). The phenotypes have been maintained throughout passages (P0, 1, 2, 3, 4, 5 . . . ) (data not shown), further confirming establishment of stable AML disease in mice and without phenotype/genotype shifts.

We then further analyzed the genotype of the leukemic cells derived in the AML mice. The results confirmed heterozygous presence of the IDH2 R140Q mutation, along with FLT3-ITD (data not shown), DNMT3A R882H and NPM1, as seen in the original AML patient leukemic cells, further confirming no genetic shift had occurred in these models.

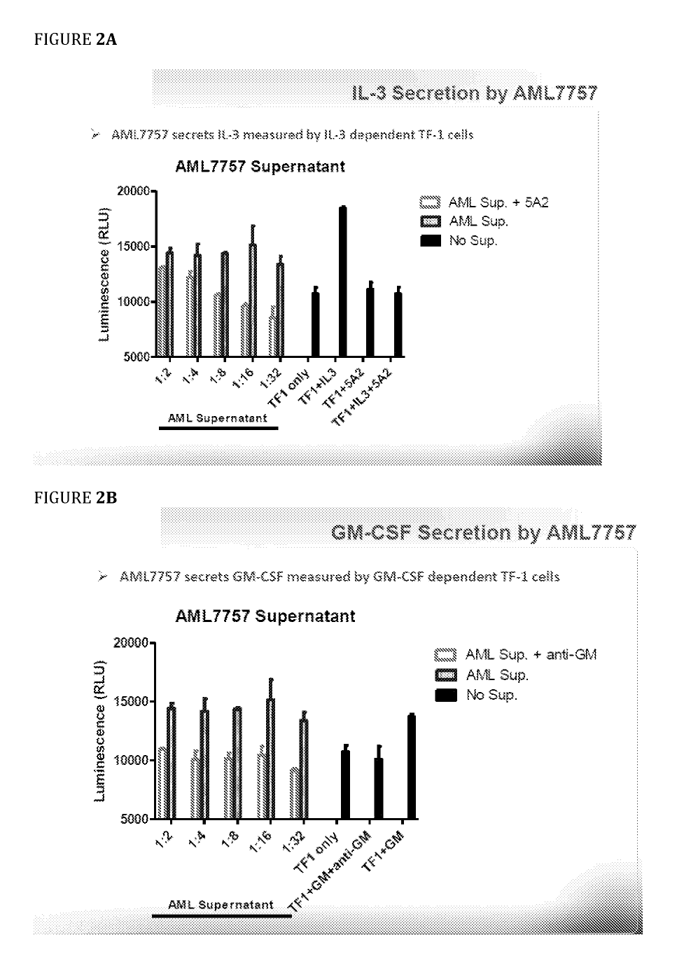

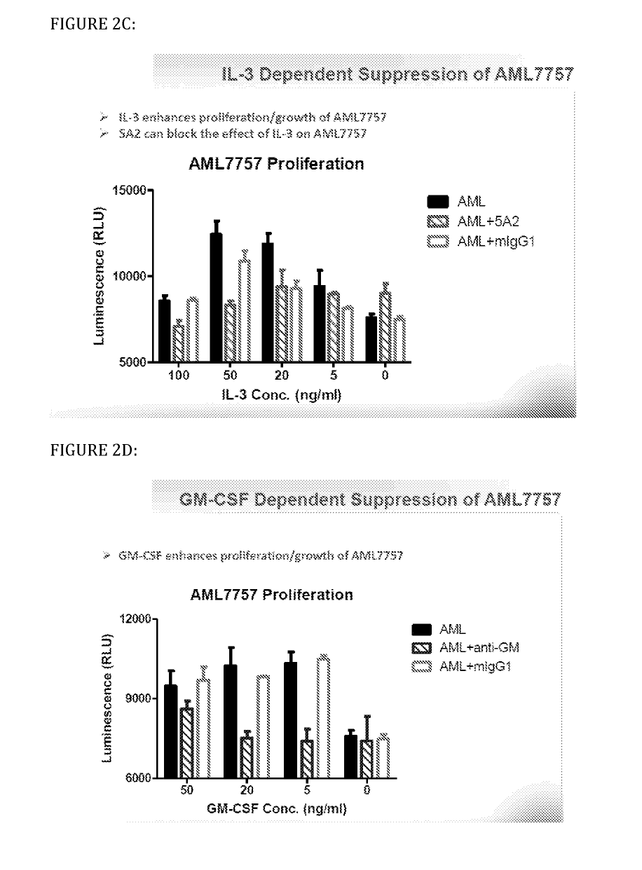

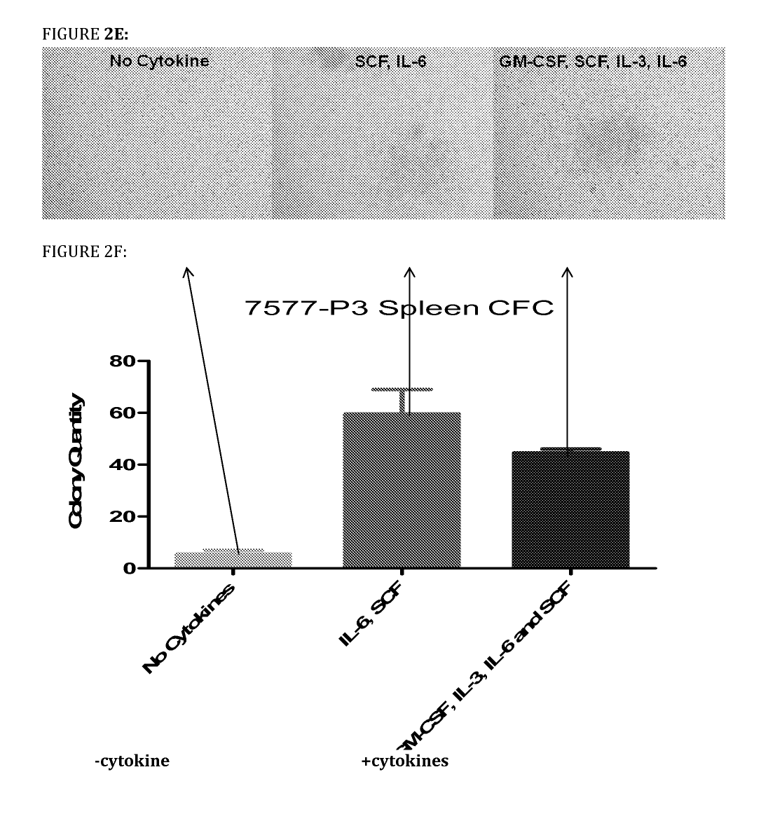

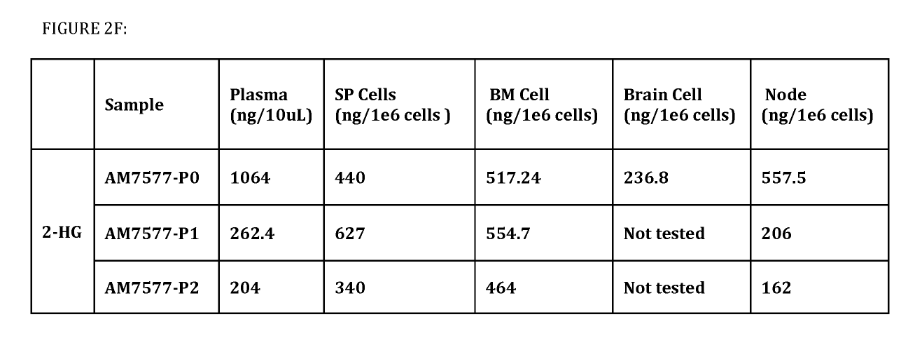

The AM7577-mice produce 2HG in leukemic cells and in mouse plasma. AML patients with IDH1 or IDH2 mutations both produce 2HG. Therefore we reasoned that we could potentially detect 2HG in AM7577-mice. By using HPLC/MS, we were able to detect and quantify 2HG in the plasma of leukemic mice and also in the leukemic cell lysate (FIG. 2G), reflective of another important character of this disease patient. We also found that the quantity of 2HG was correlated to the leukemic load in mice (FIG. 2G). We also concluded that measuring 2HG would also be reflective of the effect of drug treatment and an assessment of treatment efficacy and efficiency (FIG. 2G). This suggests that 2HG could serve as the pharmacodynamic marker for this mouse AML model.

### Example 3

### AM7577 Growth in Mice is Likely Enabled by Autocrine Mechanisms of GM-C SF and IL-3 Signaling

GM-CSF and IL-3 are two cytokines required for myeloid lineage cell growth and differentiation. They are also important for growth of many AML leukemic cells. Murine GM-CSF and IL-3 do not cross-interact with the human receptors and thus do not support the growth of many AML-patient xenografts. To this end, we assayed for the presence human GM-CSF and IL-3 in blood of AM7577-mice. Interestingly, we found high levels of human GM-CSF and IL-3 produced by the leukemic cells as shown in FIGS. 2A and 2B. Therefore, autocrine mechanisms of these two growth receptors could be an important mechanism for the AML growth of AM7577-mice, particularly at this high level, as similarly seen before (Ailles L E, et al. Blood. 1999; 94:1761-72). Both CD123 and IL-3 receptor α-chain were indeed expressed in AM7577 cells, consistent with this assumption.

We next performed short term cultures of AM7577 leukemic cells to confirm the activity of a functional role of these two cytokines. First, the supernatant of these cultures was added as exogenous cytokines to the IL-3 and GM-CSF dependent cell line TF-1 cells. As expected, these supernatants were shown to specifically stimulate TF-1 cell growth by ˜1.4 fold. Conversely, TF-1 cell growth can be suppressed by both anti-IL-3R and anti-GM-CSF antibodies, respectively. These results confirmed the production of functional GM-CSF and IL-3 by AM7577 cells.

We proceeded to test if blocking the autocrine cytokines of IL-3 and GM-CSF would indeed suppress the proliferation of AM7577 cells in vitro. AM7577 cells were cultured with±exogenous GM-CSF or IL-3, and ±anti-GM-CSF or anti-IL-3 neutralizing antibodies. As expected it was confirmed that blocking IL-3 or GM-CSF did indeed have an inhibitory effect on AM7577 cell proliferation (FIGS. 2C-2D). These results further confirmed that autocrine signals of both GM-CSF and IL-3 are participating in the growth of AM7577.

### Example 4

### CFCs in AM7577 Model Leukemic Cells

AML is a classic stem cell disease where a small subset of AML-LICs among the patient leukemic cell population are responsible for repopulating leukemic blasts of myeloid phenotypes. Similar to normal myeloid stem cells/progenitors, LICs should form colonies in the semi-liquid matrix (colony formation cells or CFCs). To this end, we tested CFC formation of AM7577-splenocytes in three sets of semi-liquid matrices: 1) without cytokine, 2) SCF+IL-6, and 3) SCF+IL-6+GM-CSF+IL-3. The results showed that only the culture in the presence of human cytokines grew and that without human cytokines neither normal mouse hematopoietic stem cells/progenitors nor LICs form colonies (FIG. 2E). The two sets with human cytokines form colonies, suggesting the presence of CFCs in the AM7577-leukemic cell population since human cytokines do not support mouse CFCs (no colony formation from non-engrafted NOD/SCID mice). In fact, the set having a full cytokine mix of IL3, IL-6, GM-CSF and SCF-1 did not lead to a higher number of colonies than the subset cytokine mix of only SCF-1 and IL-6 (FIG. 2F). The results suggest a GM-CSF/IL3-independent CFCs (with frequency around ˜0.8 CFCs/1000 cells), or LICs. This result is consistent with our assumption of an autocrine mechanism and data from in vivo engraftment titration experiments (Table 3, supra).

### Example 5

### Cytarabine (AraC) Completely Suppressed AM7577-Leukemic Development of AML but Without Cure

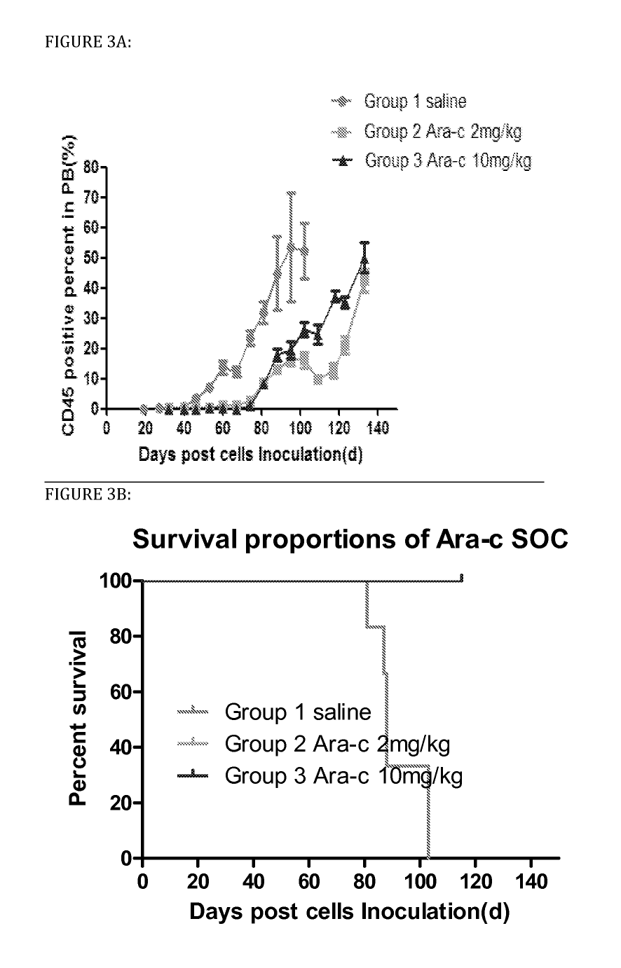

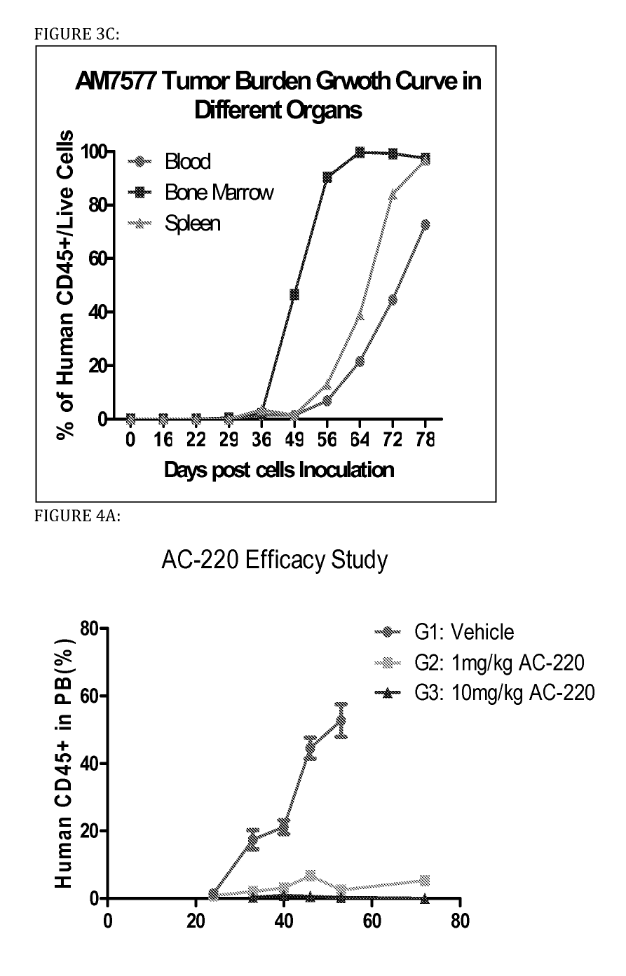

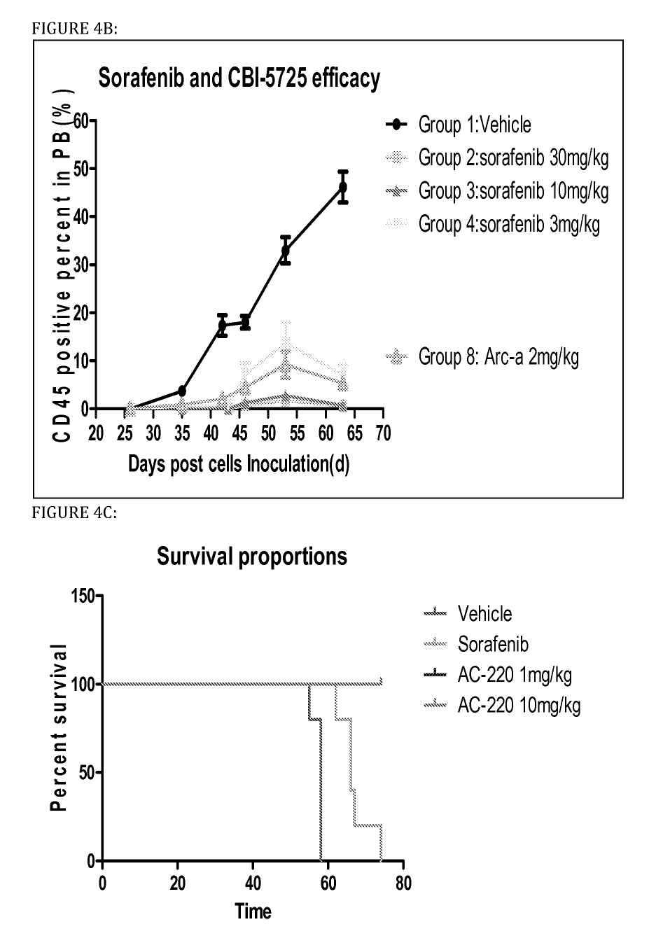

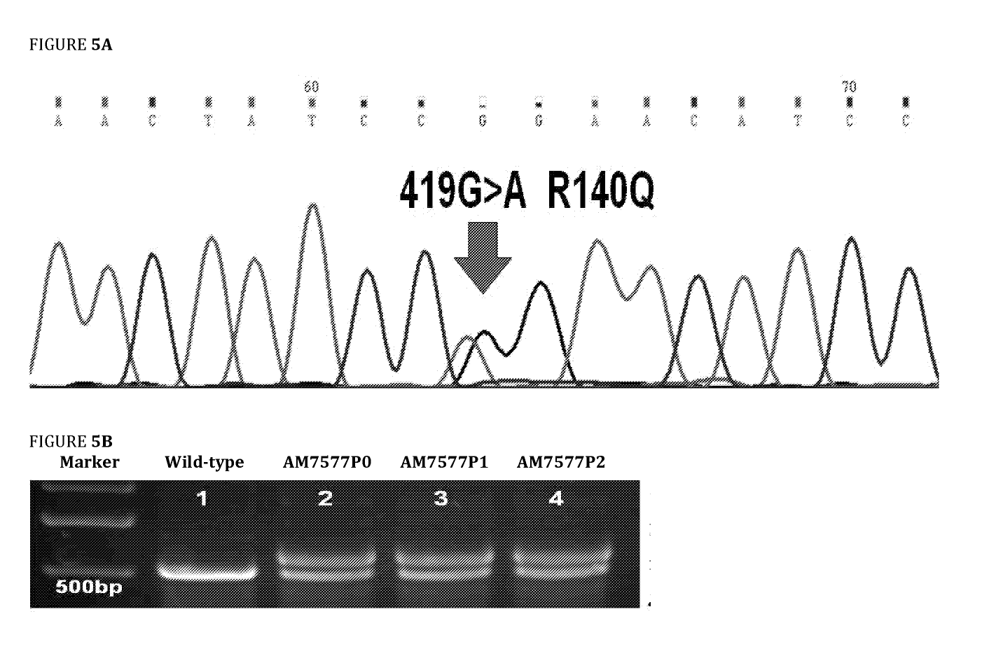

Cytarabine (AraC) is the current standard chemotherapy agent for induction therapy of AML, including AML-M5. We tested AraC anti-leukemic activity against AM7577, by subjecting AM7577-mice to a 5-day-on/2-day-off Ara-C dosing regimen, starting 15 days post-engraftment. The treated animals at both low and high dose levels (3 and 10 mg/kg) completely suppressed leukemic development as demonstrated by no detectable leukemic load in peripheral blood, as opposed to the steady increase of leukemic cells (CD45⁺) starting from 30 days post-engraftment in the vehicle group (FIG. 3A). The suppression of tumor growth was also translated into extended AM7577-mice survival times (FIG. 3B).

We subsequently explored whether AraC treatment actually cured the disease by withdrawing treatment. The result was rapid relapse just a few days post-treatment withdrawal (FIG. 3A). This observation can potentially be interpreted as AraC has only affected leukemic blasts, but has no affect on LICs. The appearance of leukemic blasts in peripheral blood after AraC withdrawal can be effectively re-suppressed by resuming AraC treatment (FIG. 3A).

While various embodiments of the present invention have been described in detail, it is apparent that modifications, adaptations and equivalents of those embodiments will occur to those skilled in the art. It is to be expressly understood, however, that such modifications and adaptations are within the scope of the present invention and are intended to be encompassed herein, as set forth in the following claims.

### REFERENCES

Agliano A, Martin-Padura I, Mancuso P, Marighetti P, Rabascio C, Pruneri G, et al. Human acute leukemia cells injected in NOD/LtSz-scid/IL-2Rgamma null mice generate a faster and more efficient disease compared to other NOD/scid-related strains. Int J Cancer. 2008 (123):2222-7.

Ailles L E, Gerhard B, Kawagoe H, Hogge D E. Growth chteristics of acute myelogenous leukemia progenitors that initiate malignant hematopoiesis in nonobese diabetic/severe combined immunodeficient mice. Blood. 1999; 94:1761-72.

Dang L, Jin S, Su S M. IDH mutations in glioma and acute myeloid leukemia. Trends Mol Med. 2010; 16:387-97.

Feuring-Buske M, Gerhard B, Cashman J, Humphries R K, Eaves C J, Hogge D E. Improved engraftment of human acute myeloid leukemia progenitor cells in beta 2-microglobulin-deficient NOD/SCID mice and in NOD/SCID mice transgenic for human growth factors. Leukemia. 2003 (17):760-3.

Lumkul R, Gorin N C, Malehorn M T, Hoehn G T, Zheng R, Baldwin B, et al. Human AML cells in NOD/SCID mice: engraftment potential and gene expression. Leukemia. 2002 (16):1818-26.

Malaise M, Neumeier M, Botteron C, Dohner K, Reinhardt D, Schlegelberger B, et al. Stable and reproducible engraftment of primary adult and pediatric acute myeloid leukemia in NSG mice. Leukemia. 2011; 25:1635-9.

Pearce D J, Taussig D, Zibara K, Smith L L, Ridler C M, Preudhomme C, et al. AML engraftment in the NOD/SCID assay reflects the outcome of AML: implications for our understanding of the heterogeneity of AML. Blood. 2006 (107):1166-73.

Rombouts W J, Martens A C, Ploemacher R E. Identification of variables determining the engraftment potential of human acute myeloid leukemia in the immunodeficient NOD/SCID human chimera model. Leukemia. 2000 (14):889-97.

Rombouts W J, Blokland I, Lowenberg B, Ploemacher R E. Biological characteristics and prognosis of adult acute myeloid leukemia with internal tandem duplications in the Flt3 gene. Leukemia. 2000 (14):675-83.

Shultz L D, Lyons B L, Burzenski L M, Gott B, Chen X, Chaleff S, et al. Human lymphoid and myeloid cell development in NOD/LtSz-scid IL2R gamma null mice engrafted with mobilized human hemopoietic stem cells. J Immunol. 2005; 174:6477-89.

Yang M, Baoen Shan, Qiaoxia Li, Xiaoming Song, Jianyun Deng, Jie Cai, Likun Zhangl, Junjie Lu, Zhenjian Du, Taiping Chen, Jean-Pierre Wery, Yiyou Chen and Qixiang Li. Overcoming erlotinib resistance with tailored treatment regimen in patient derived xenografts from naïve Asian NSCLC patients. Int J Cancer. 2013, E74-E84; September 5. doi: 10.1002/ijc.27813. [Epub ahead of print].

Zhou K G, Jiang L J, Shang Z, Wang J, Huang L, Zhou J F. Potential application of IDH1 and IDH2 mutations as prognostic indicators in non-promyelocytic acute myeloid leukemia: a meta-analysis. Leuk Lymphoma. 2012, 2012 53(12):2423-9. doi: 10.3109/10428194.2012.695359. Epub 2012 Jun. 18.

It is claimed:

1. A method of screening for a therapeutic agent for human acute myelogenous leukemia, comprising a) a step of administering a test substance to an immunocompromised NOD/SCID mouse engrafted with a substance containing a leukemic cell derived from a human acute myelogenous leukemia patient and b) a step of assessing improvement in leukemia in the mouse.

2. The method according to claim 1, further comprising c) a step of monitoring a side effect of the test substance in the mouse.

3. The method according to claim 2, comprising examining the peripheral blood collected from the mouse in step b) and/or step c).

4. The method according to claim 1, which reproduces in the mouse the pathology of leukemia in the patient from whom the substance is derived.

5. A mouse having selectively expanded human leukemic cells, which are obtained by the method according to claim 1.

6. The mouse according to claim 5, wherein the leukemic cells are leukemia initiation cells (LICs).

7. The mouse according to claim 5, wherein the mouse is a leukemic mouse.

8. The mouse according to claim 7, wherein the mouse exhibits at least one of an enlarged spleen or the presence of leukemic cells.

9. The mouse according to claim 8, wherein the leukemic cells are present in at least one of bone marrow or a peripheral organ such as spleen or blood.

10. The method according to claim 7, wherein the leukemic mouse is an acute myelogenous leukemic mouse.

11. The method according to claim 1, wherein engraftment of the mouse occurs when the mouse is about 4 weeks of age.

12. The method according to claim 1, wherein the engrafted cells test positive for production of at least one of GM-CSF and IL-3.

13. The mouse according to claim 10, wherein the pathology of leukemia in the mouse is selected from human AML subtype M0, M1, M2, M3, M4, M5, M6, and M7.

14. The method according to claim 13, wherein the patient further comprises a heterozygous mutation.

15. The method according to claim 14, wherein the mutation comprises an IDH2 mutation.

16. The method according to claim 15, wherein the IDH2mutation comprises an R140Q mutation.

17. A method of selecting or optimizing a method of treating a patient from whom leukemic cells in the peripheral blood are derived from an immunocompromised NOD/SCID mouse engrafted with a substance containing a leukemic cell derived from a human acute myelogenous leukemia patient comprising a) a step of providing the mouse with a treatment of human acute myelogenous leukemia, and b) a step of assessing an improvement and/or a side effect caused by the treatment of leukemia in the mouse.

18. The method of claim 17, wherein a mouse having human leukemic cells in peripheral blood is derived from a different non-adult immunocompromised NOD/SCID mouse about 4 weeks old having one or more repeats of a step of engrafting a substance containing a leukemic cell derived from the mouse obtained by the method according to claim 17 and raising the different mouse, wherein the different mouse has human leukemic cells in at least one of the mouse peripheral blood, bone marrow or spleen.

19. A method of producing a mouse having human leukemic cells, comprising engrafting a substance containing a leukemic cell derived from a human acute myelogenous leukemia patient into a non-adult immunocompromised NOD/SCID mouse and raising the mouse.

20. A method of producing a mouse having human leukemic cells, comprising one or more repeats of a step of engrafting a substance containing a leukemic cell derived from the mouse obtained by the method according to claim 19 into a different non-adult immunocompromised NOD/SCID mouse and raising the mouse.
